(19) United States
(12) Patent Application Publication
(10) Pub. No.: US 2024/0285469 A1
(43) Pub. Date: Aug. 29, 2024

(54) BALANCE REHABILITATION APPARATUS

(71) Applicants: Hsiu-Chung Ou, Taichung (TW); Ears on Technology Co., Ltd., Taichung City (TW)

(72) Inventors: Shin-Da LEE, Taichung (TW); Hsiu-Chung OU, Taichung (TW)

(21) Appl. No.: 18/115,504

(22) Filed: Feb. 28, 2023

(51) Int. Cl. A61H 23/02; A61N 1/26; A61N 2/00; A61N 2/02; A61N 5/06; A63B 22/18

(52) U.S. Cl. CPC A61H 23/0236; A61H 23/02; A61N 1/26; A61N 2/002; A61N 2/02; A61N 5/0618; A63B 22/18; A61H 2201/0173; A61H 2201/10; A61H 2201/1604; A61H 2201/5005; A61H 2201/5043; A61H 2205/027

(57) ABSTRACT

A balance rehabilitation apparatus is provided, including an audio stimulation unit, an electronic stimulation unit and a control unit. The audio stimulation unit includes two speakers, and the electronic stimulation unit includes a plurality of first electronic stimulation agents and two second electronic stimulation agents. Two of the plurality of first electronic stimulation agents correspond to cheeks of a user, two of the plurality of first electronic stimulation agents correspond to a cerebellum of the user, and the two second electronic stimulation agents correspond to two mastoid processes of the user. The control unit is electrically connected with the two speakers, the plurality of first electronic stimulation agents and the two second electronic stimulation agents and simultaneously controls the two speakers, the plurality of first electronic stimulation agents and the two second electronic stimulation agents to operate to increase the efficiency of balance rehabilitation.

### BACKGROUND OF THE INVENTION

### Field of the Invention

The present invention relates to a balance rehabilitation apparatus.

### Description of the Prior Art

According to the literature statistics, an average of one-third of the elderly people fall at least once a year due to balance problems, which may cause the elderly people to be disabled and affect their quality of life, and also cause a large medical burden. Therefore, it is necessary and important to find a rehabilitation system for the elderly people to improve their balance ability.

The present invention is, therefore, arisen to obviate or at least mitigate the above-mentioned disadvantages.

### SUMMARY OF THE INVENTION

The main object of the present invention is to provide a balance rehabilitation apparatus.

To achieve the above and other objects, the present invention provides a balance rehabilitation apparatus configured for a user to use. The balance rehabilitation apparatus includes a support unit, an audio stimulation unit, an electronic stimulation unit and a control unit.

The support unit corresponds to a head shape of the user and includes two ears portions corresponding to ears of the user and a top-side portion connected between the two ears portions and configured to be disposed across a top of a head of the user upwardly. The audio stimulation unit includes two speakers disposed on the two ears portions, and the two speakers are configured to play binaural beats with frequency following response to the ears of the user. The binaural beats with frequency following response has an audio frequency difference with gradually change of frequency difference. The electronic stimulation unit includes two front extension pieces connected with the two ears portions and extending to cheeks of the user respectively, a rear extension piece connected between the two ears portions and configured to be disposed across a back of the head of the user, a plurality of first electronic stimulation agents and two second electronic stimulation agents. Two of the plurality of first electronic stimulation agents are respectively disposed on the two front extension pieces for corresponding to the cheeks of the user, and two of the plurality of first electronic stimulation agents are disposed on the rear extension piece for corresponding to two sides of the user cerebellum. The two second electronic stimulation agents are disposed on an inner side of the rear extension piece for corresponding to two mastoid processes of the user. The plurality of first electronic stimulation agents and the two second electronic stimulation agents are configured to output a physical stimulation including one of a current for transcranial electrical stimulation and an electromagnetic pulse for transcranial magnetic stimulation to the head of the user. The control unit is electrically connected with the two speakers, the plurality of first electronic stimulation agents and the two second electronic stimulation agents. The control unit stores digital information of the binaural beats with frequency following response and controls the two speakers to play the binaural beats with frequency following response simultaneously, and controls the plurality of first electronic stimulation agents and the two second electronic stimulation agents to output the physical stimulation.

The advantage of the present invention is that: with the two speakers, the plurality of first electronic stimulation agents and the two second electronic stimulation agents, the user receives multiple stimuli in a single period of treatment, which activates the cerebellum of the user and effectively improves the efficiency of balance rehabilitation.

The present invention will become more obvious from the following description when taken in connection with the accompanying drawings, which show, for purpose of illustrations only, the preferred embodiment(s) in accordance with the present invention.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 is a stereogram of a first preferable embodiment of the present invention;

FIG. 2 is another stereogram of the first preferable embodiment of the present invention as viewed from another side;

FIG. 3 is a block diagram of the first preferable embodiment of the present invention;

FIG. 4 is a partial front view according to the first preferable embodiment of the present invention;

FIG. 5 is a partial side view according to the first preferable embodiment of the present invention;

FIG. 6 is a partial rear view according to the first preferable embodiment of the present invention;

FIG. 7 is a magnetic resonance image of a user after listening to a binaural beats with frequency following response;

FIG. 8 is a magnetic resonance image of the user after listening to another binaural beats with frequency following response;

FIG. 9 is a stereogram of a second preferable embodiment of the present invention;

FIG. 10 is a block diagram of the second preferable embodiment of the present invention;

FIG. 11 is a magnetic resonance image of the user after receiving an optical frequency-flashed stimulation;

FIG. 12 is a magnetic resonance image of the user after receiving another optical frequency-flashed stimulation;

FIG. 13 is a stereogram of a third preferable embodiment of the present invention; and

FIG. 14 is a stereogram of a fourth preferable embodiment of the present invention.

### DETAILED DESCRIPTION OF THE PREFERRED EMBODIMENTS

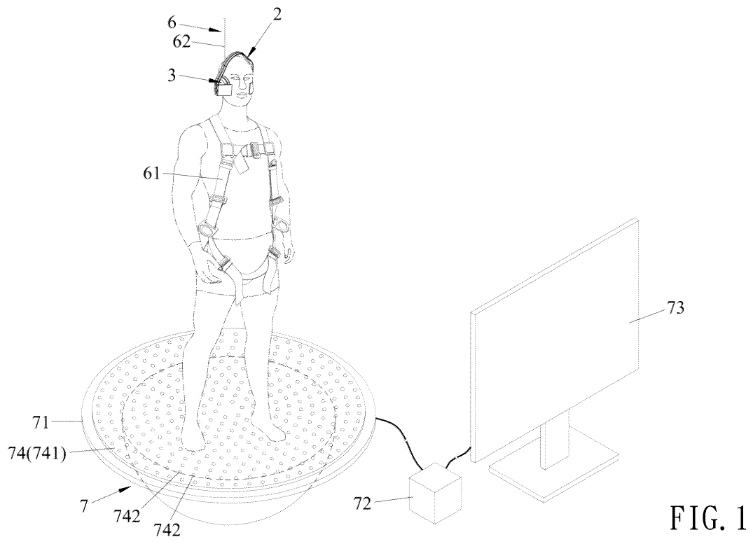

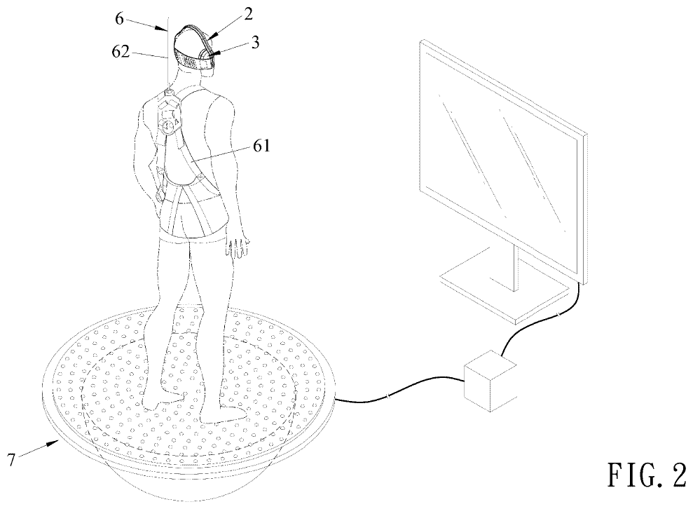

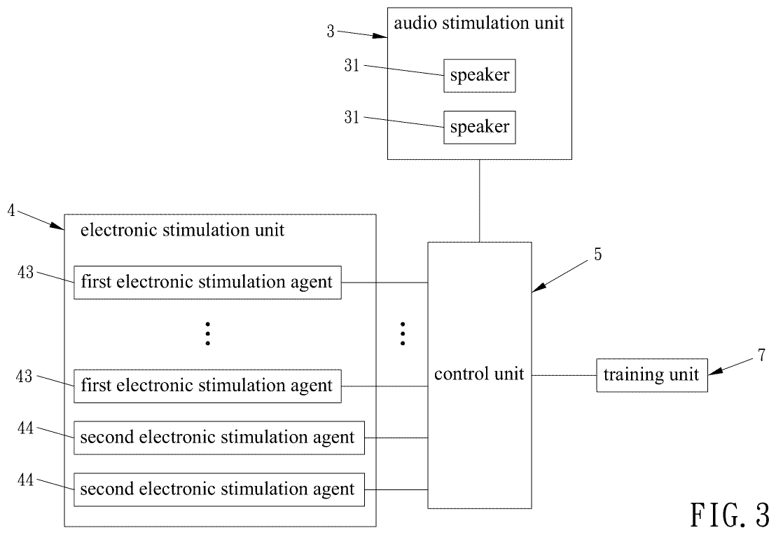

Please refer to FIGS. 1 to 3 for a first preferable embodiment of the present invention. A balance rehabilitation apparatus of the present invention is configured for a user to use. The balance rehabilitation apparatus includes a support unit 2, an audio stimulation unit 3, an electronic stimulation unit 4, a control unit 5, a protection unit 6 and a training unit 7.

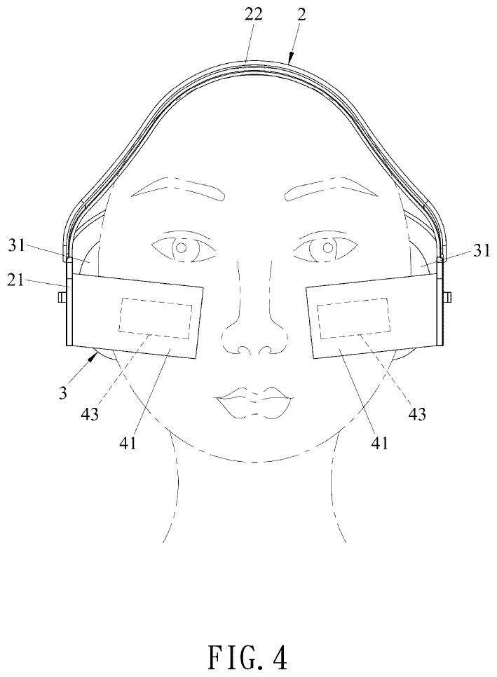

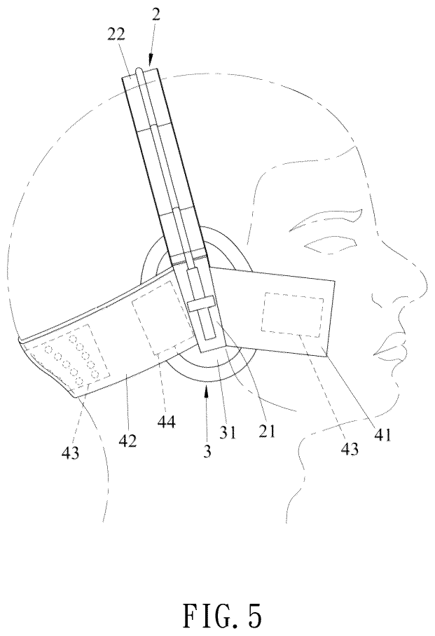

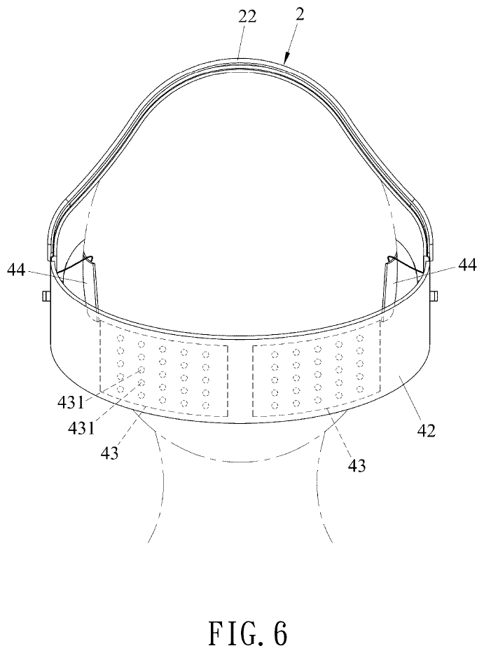

Please refer to FIGS. 4, 5 and 6, the support unit 2 corresponds to a head shape of the user and includes two ears portions 21 corresponding to ears of the user and a top-side portion 22 connected between the two ears portions 21 and configured to be disposed across a top of a head of the user upwardly.

The audio stimulation unit 3 includes two speakers 31 disposed on the two ears portions 21, and the two speakers 31 are configured to play a binaural beats with frequency following response to the ears of the user. The binaural beats with frequency following response has an audio frequency difference. In this embodiment, the audio frequency difference of the binaural beats with frequency following response is from 29 Hz to 16 Hz so as to activate a cerebellum (balance center) of the user.

The electronic stimulation unit 4 includes two front extension pieces 41 connected with the two ears portions 21 and extending to cheeks of the user respectively, a rear extension piece 42 connected between the two ears portions 21 and configured to be disposed across a back of the head of the user, four first electronic stimulation agents 43 and two second electronic stimulation agents 44.

Two of the plurality of first electronic stimulation agents 43 are respectively disposed on the two front extension pieces 41 for corresponding to the cheeks of the user and are used as reference electrodes (cathode). Two of the plurality of first electronic stimulation agents 43 are disposed on the rear extension piece 42 for corresponding to two sides of the cerebellum of the user and are used as stimulating electrodes (anode). The two second electronic stimulation agents 44 are disposed on an inner side of the rear extension piece 42 for corresponding to mastoid processes behind the ears of the user. In this embodiment, the plurality of first electronic stimulation agents 43 and the two second electronic stimulation agents 44 are configured to output physical stimulations which are transcranial direct current stimulations (tDCS); the current intensity is between 0.5 mA and 2 mA, and the current density is between 0.03 mA/cm² and 0.09 mA/cm². In other embodiments, the physical stimulations may be electromagnetic pulses for transcranial magnetic stimulations (TMS), and the electromagnetic frequency of the electromagnetic pulses is between 1 Hz and 20 Hz.

Two of the plurality of first electronic stimulation agents 43 disposed on the rear extension piece 42 and corresponding to the cerebellum of the user include a plurality of conductive pillars 431 parallel to one another and configured to output the physical stimulations.

Please refer to FIGS. 1, 3 and 5, the control unit 5 is electrically connected with the two speakers 31, the plurality of first electronic stimulation agents 43 and the two second electronic stimulation agents 44. The control unit 5 stores digital information of the binaural beats with frequency following response and controls the two speakers 31 to play the binaural beats with frequency following response simultaneously and the plurality of first electronic stimulation agents 43 and the two second electronic stimulation agents 44 to output the physical stimulations. In this embodiment, the control unit 5 is a personal computer.

The protection unit 6 includes a safety strap 61 configured for the user to wear and a fixed strap 62 connected with the safety strap 61 and configured to be connected with a fixed object above the user. In this embodiment, the safety strap 61 is similar to the protective gear used in mountaineering or rock climbing.

The training unit 7 includes a balance board 71 configured for the user to stand thereon, a control box 72 electrically connected with the balance board 71, a monitor 73 electrically connected with the control box 72, and a foot stimulation pad 74 configured for the user to step thereon.

The balance board 71 outputs a sense signal corresponding to a posture of the user, and the control box 72 receives the sense signal and controls the monitor 73 to display an image corresponding to the sense signal.

The foot stimulation pad 74 includes a base portion 741 detachably disposed on the balance board 71 and a plurality of foot stimulation portions 742 protruding upward from the base portion 741.

In operation, the user wears the support unit 2 and the protection unit 6 and stands on the balance board 71 through the foot stimulation pad 74 with the safety aid of the safety strap 61 and the fixed strap 62. Then, the control unit 5 controls the two speakers 31 to play the binaural beats with frequency following response and controls the plurality of first electronic stimulation agents 43 and the two second electronic stimulation agents 44 to output the physical stimulations to corresponding brain region. Therefore, the user receives stimulations of the binaural beats with frequency following response and the physical stimulations at the same time, and receives proprioceptive stimulations from the foot stimulation portion 742 of the foot stimulation pad 74.

When the user changes posture on the balance board 71, the control box 72 controls the monitor 73 to show corresponding image according to the sense signal of the balance board 71 so that the user can manipulate the images on the monitor 73 by changing his actions. In this embodiment, the monitor 73 may show a maze, and the user can change posture on the balance board 71 to move in the maze so as to train the balance ability of the user.

Specifically, the test results corresponding to the physical stimulations of the plurality of first electronic stimulation agents 43 and the two second electronic stimulation agents 44 on balance rehabilitation have been reported by Fujimoto, C., et al., Noisy galvanic vestibular stimulation induces a sustained improvement in body balance in elderly adults (Sci Rep, 2016. 6: p. 37575)
and Goel, R., et al., Using Low Levels of Stochastic Vestibular Stimulation to Improve Balance Function (PLOS One, 2015. 10(8): p. e0136335). According to Corbin, D. M., et al., The effect of textured insoles on postural control in double and single limb stance (J Sport Rehabil, 2007. 16(4): p. 363-72) and Annino, G., et al., The efficacy of plantar stimulation on human balance control (Somatosens Mot Res, 2015. 32(3): p. 200-5), standing on insoles with protruding granules can increase proprioceptive stimulation and improve the balance ability.

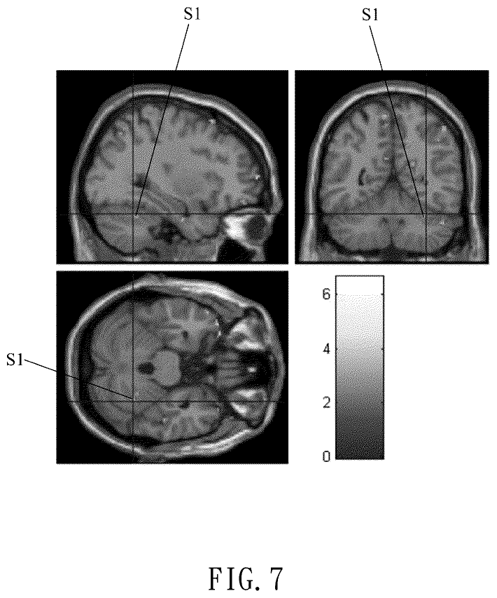

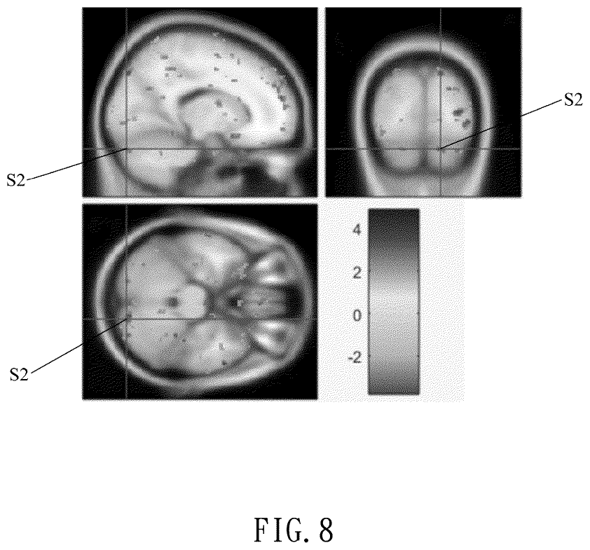

Furthermore, please refer to FIG. 7, showing a magnetic resonance image (MRI) taken from a subject after listening to the binaural beats with frequency following response with the audio frequency difference of 16-29 Hz. Activation occurs at the coordinates (79,95,68) positioned by MRI (the yellow-orange activation area S1 at the blue cross in FIG. 7). Please refer to FIG. 8, showing a magnetic resonance image (MRI) taken from another subject after listening to the binaural beats with frequency following response with the audio frequency difference of 25-29 Hz. Activation occurs at the coordinates positioned by MRI (the yellow-orange activation area S2 at the blue cross in FIG. 8). The activation areas S1, S2 correspond to the balance area of the brain, which proves that listening to the binaural beats with frequency following response with the audio frequency difference can have an impact to the balance area of the brain.

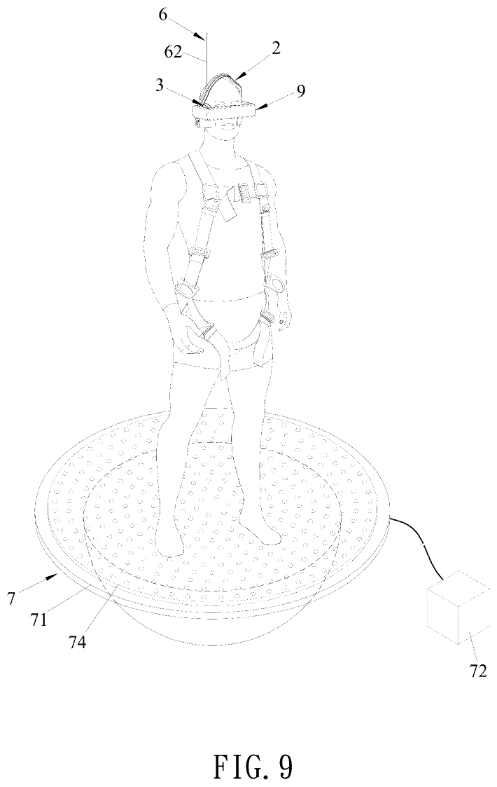

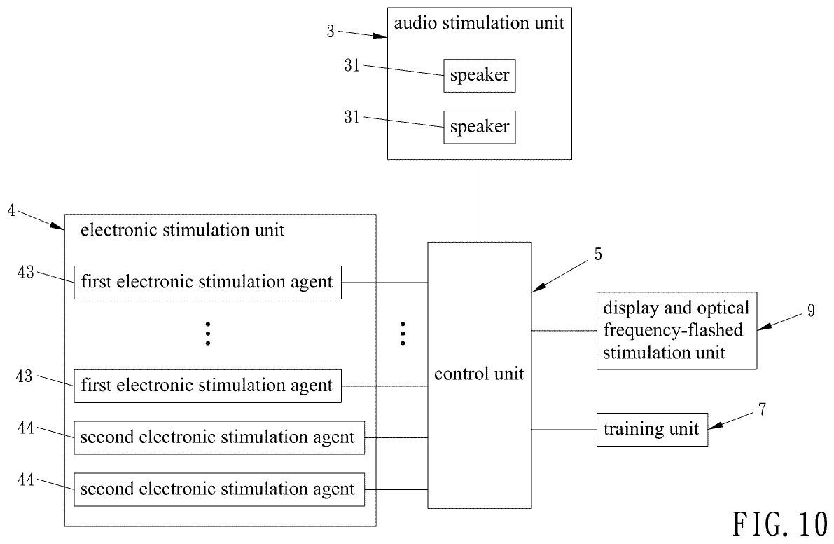

Please refer to FIGS. 9 and 10, showing a second preferable embodiment of the present invention, which differs from the first preferable embodiment in that:

The training unit 7 includes the balance board 71, the control box 72 and the foot stimulation pad 74.

The balance rehabilitation apparatus further includes a display and optical frequency-flashed stimulation unit 9. The display and optical frequency-flashed stimulation unit 9 is disposed on the support unit 2 and electrically connected with the control unit 5, and the display and optical frequency-flashed stimulation unit 9 is communicatively connected with the control box 72. In this embodiment, the display and optical frequency-flashed stimulation unit 9 is a virtual image monitor.

The display and optical frequency-flashed stimulation unit 9 is controllable by the control unit 5 to be switched between a display mode, a training mode and a frequency-flashed stimulation mode.

In the display mode and the training mode, the display and optical frequency-flashed stimulation unit 9 displays a virtual image to the user; and in the frequency-flashed stimulation mode, the display and optical frequency-flashed stimulation unit 9 displays a flashing image to stimulate eyes of the user. In this embodiment, the virtual image is a VR image.

In the display mode, the virtual image shows the sense signal corresponding to the balance board 71, and the virtual image and the display and optical frequency-flashed stimulation unit 9 are moved in the same coordinate system. The virtual image is co-moved with the display and optical frequency-flashed stimulation unit 9 and the user does no feel movement of the virtual image when the display and optical frequency-flashed stimulation unit 9 is moved.

In the training mode, the virtual image and the display and optical frequency-flashed stimulation unit 9 are moved in different coordinate systems. The coordinates of the virtual image is not moved and the user feels the virtual image moving in an opposite direction relative to the display and optical frequency-flashed stimulation unit 9 when the display and optical frequency-flashed stimulation unit 9 is moved. In this embodiment, the virtual image shows a fixed object, since the coordinates of the virtual image is not moved, the user can gaze at the fixed object when the user quickly turns his head away from the fixed object. By repeating gaze training back and forth, vestibular functions of the user are trained so as to improve the balance ability.

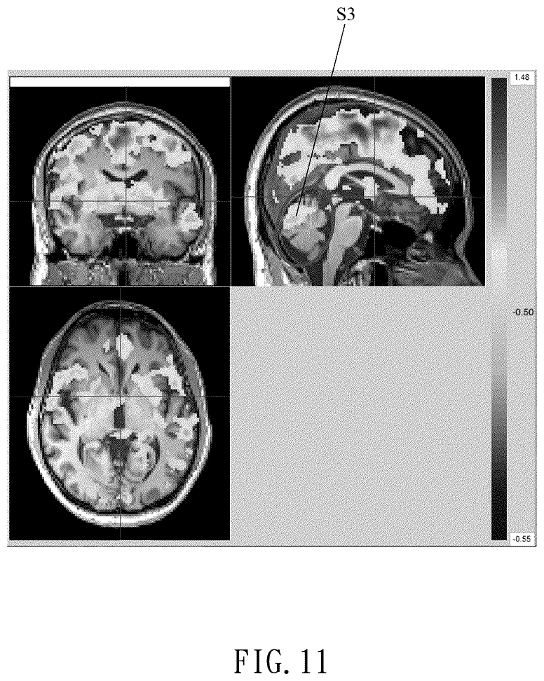

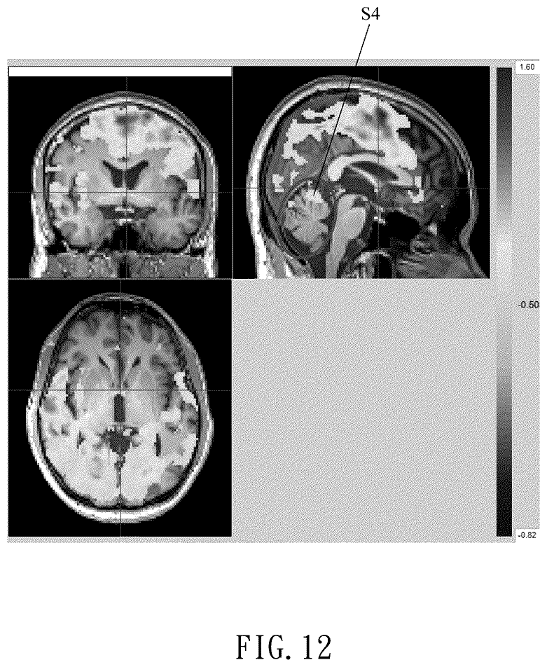

Moreover, stimulating the user's eyes with a flashing image may improve the balance ability of the user. In this embodiment, when the display and optical frequency-flashed stimulation unit 9 is in the frequency-flashed stimulation mode, the flashing image displayed by the display and optical frequency-flashed stimulation unit 9 is flashed with a predetermined optical frequency, and the predetermined optical frequency is between 25 Hz and 50 Hz. Please refer to FIG. 11, showing a magnetic resonance image taken from a subject after receiving the optical frequency-flashed stimulation with the predetermined optical frequency of 25 Hz. Activation occurs at a position corresponding to the brain of the subject positioned by MRI (the yellow-orange activation area S3 inside the red circle in FIG. 11). Please refer to FIG. 12, showing a magnetic resonance image taken from f a subject after receiving the optical frequency-flashed stimulation with the predetermined optical frequency of 40 Hz. Activation occurs at a position corresponding to the brain of the subject positioned by MRI (the yellow-orange activation area S4 inside the red circle in FIG. 12). The activation areas S3, S4 correspond to the functional connection between the motor cortex and the cerebellum, which proves that the optical frequency-flashed stimulation can have an impact on balance. In other embodiments, when the display and optical frequency-flashed stimulation unit 9 is in the frequency-flashed stimulation mode, the display and optical frequency-flashed stimulation unit 9 displays an flashing image with an optical frequency difference to stimulate the eyes of the user respectively.

Therefore, the second preferable embodiment can achieve the same purpose and effect as the first preferable embodiment mentioned above.

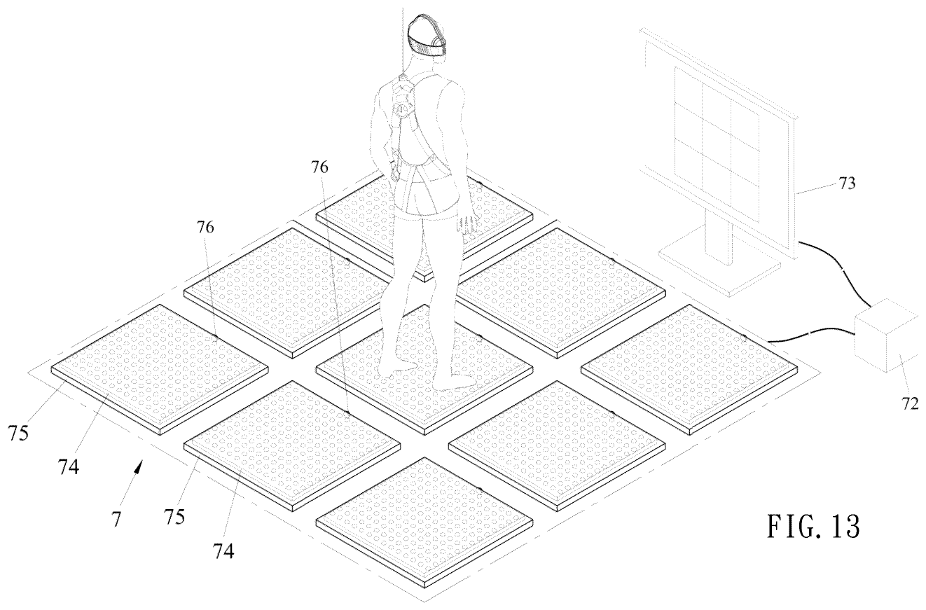

Please refer to FIG. 13, showing a third preferable embodiment of the present invention, which differs from the first preferable embodiment in that:

The training unit 7 includes nine stepping boards 75 arranged spacedly on a ground in an array and configured for the user to stepped thereon, nine indicator lights 76 respectively disposed on the nine stepping boards 75, the control box 72 electrically connected with the nine stepping boards 75 and the nine indicator lights 76, the monitor 73 electrically connected with the control box 72, and nine foot stimulation pads 74 respectively disposed on the nine stepping boards 75.

Each of the nine stepping boards 75 outputs a stepping signal corresponding to respective stepped states, and the control box 72 receives said stepping signals and controls the monitor 73 to display an image corresponding to the said stepping signals.

In this embodiment, the image is a nine-square grid image corresponding to the nine stepping boards 75, and the control box 72 controls one of the nine indicator lights 76 to light up. Therefore, the user steps on one of the nine stepping boards 75 via corresponding one of the nine foot stimulation pads 74 according to lighting one of the nine indicator lights 76, and the control box 72 controls the monitor 73 to show whether the user steps corresponding stepping boards 75 correctly so as to train the balance ability of the user.

Therefore, the third preferable embodiment can achieve the same purpose and effect as the first preferable embodiment mentioned above.

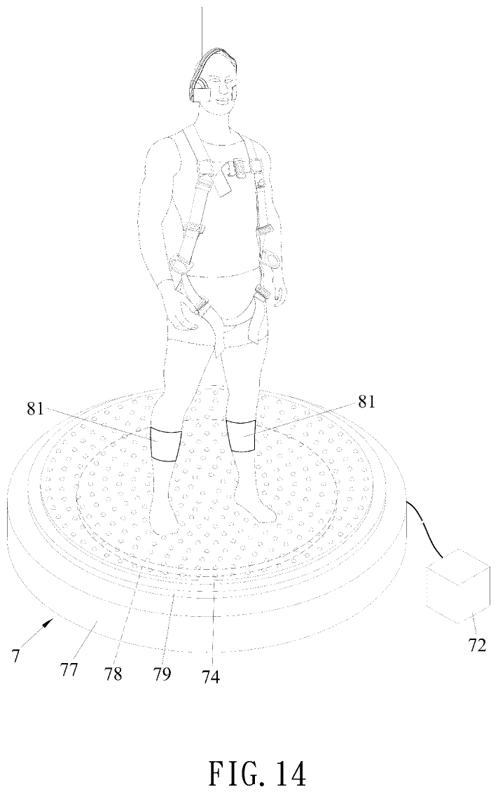

Please refer to FIG. 14, showing a fourth preferable embodiment of the present invention, which differs from the first preferable embodiment in that:

The training unit 7 includes a base 77, a vibration module 78 disposed on the base 77, a vibration board 79 disposed on the vibration module 78, the foot stimulation pad 74 disposed on the vibration board 79, the control box 72 electrically connected with the vibration module 78, and two vibration belts 81 generating vibrations and configured to be secured to the user. The vibration board 79 is drivable to vibrate by the vibration module 78 and configured for the user to stand thereon, and vibration frequencies of the vibration board 79 and the vibration belt 81 are respectively between 25 Hz and 250 Hz. In this embodiment, the two vibration belts 81 are configured to be secured to calves of the user.

According to Chien, J. H., et al., Tactile stimuli affect long-range correlations of stride interval and stride length differently during walking (Exp Brain Res, 2017. 235(4): p. 1185-1193) and Toosizadeh, N., J. Mohler, and V. Marlinski, Low intensity vibration of ankle muscles improves balance in elderly persons at high risk of falling (PLOS One, 2018. 13(3): p. e0194720), vibrating the soles or muscles can improve balance ability. Therefore, this embodiment can further provide vibration to improve balance ability.

Therefore, the fourth preferable embodiment can achieve the same purpose and effect as the first preferable embodiment mentioned above.

In summary, with the two speakers 31, the plurality of first electronic stimulation agents 43, the two second electronic stimulation agents 44, the training unit 7 and the display and optical frequency-flashed stimulation unit 9, the user receives multiple stimuli in a single period of treatment, which activates the cerebellum of the user and effectively improves the efficiency of balance rehabilitation.

Although particular embodiments of the invention have been described in detail for purposes of illustration, various modifications and enhancements may be made without departing from the spirit and scope of the invention. Accordingly, the invention is not to be limited except as by the appended claims.

What is claimed is:

1. A balance rehabilitation apparatus, configured for a user to use, including:
a support unit, corresponding to a head shape of the user, including two ears portions corresponding to ears of the user and a top-side portion connected between the two ears portions and configured to be disposed across a top of a head of the user upwardly;
an audio stimulation unit, including two speakers disposed on the two ears portions, the two speakers configured to playbinaural beats with frequency following response to the ears of the user, the binaural beats with frequency following response having an audio frequency difference;
an electronic stimulation unit, including two front extension pieces connected with the two ears portions and extending to cheeks of the user respectively, a rear extension piece connected between the two ears portions and configured to be disposed across a back of the head of the user, a plurality of first electronic stimulation agents and two second electronic stimulation agents, two of the plurality of first electronic stimulation agents respectively disposed on the two front extension pieces for corresponding to the cheeks of the user, two of the plurality of first electronic stimulation agents disposed on the rear extension piece for corresponding to two sides of a cerebellum of the user, the two second electronic stimulation agents disposed on an inner side of the rear extension piece for corresponding to mastoid processes behind the ears of the user, the plurality of first electronic stimulation agents and the two second electronic stimulation agents configured to output physical stimulations including one of a current for transcranial electrical stimulation and an electromagnetic pulse for transcranial magnetic stimulation to the head of the user; and
a control unit, electrically connected with the two speakers, the plurality of first electronic stimulation agents and the two second electronic stimulation agents, the control unit storing digital information of the binaural beats with frequency following response, controlling the two speakers to play the binaural beats with frequency following response simultaneously, and controlling the plurality of first electronic stimulation agents and the two second electronic stimulation agents to output the physical stimulations.

2. The balance rehabilitation apparatus of claim 1, wherein the audio frequency difference of the binaural beats with frequency following response is from 29 Hz to 16 Hz.

3. The balance rehabilitation apparatus of claim 1, wherein when the plurality of first electronic stimulation agents and the two second electronic stimulation agents are of a type outputting direct current, a current intensity of the current is between 0.5 mA and 2 mA, a current density of the current is between 0.03 mA/cm² and 0.09 mA/cm²; and when the plurality of first electronic stimulation agents are of a type outputting the electromagnetic pulse, an electromagnetic frequency of the electromagnetic pulses is between 1 Hz and 20 Hz.

4. The balance rehabilitation apparatus of claim 1, wherein two of the plurality of first electronic stimulation agents disposed on the rear extension piece and corresponding to the cerebellum of the user include a plurality of conductive pillars parallel to one another and configured to output the physical stimulations.

5. The balance rehabilitation apparatus of claim 1, further including a display and optical frequency-flashed stimulation unit, wherein the display and optical frequency-flashed stimulation unit is disposed on the support unit and electrically connected with the control unit, the display and optical frequency-flashed stimulation unit is switchable between a display mode and a frequency-flashed stimulation mode; in the display mode, the display and optical frequency-flashed stimulation unit displays a virtual image to the user; and in the frequency-flashed stimulation mode, the display and optical frequency-flashed stimulation unit displays a flashing image to stimulate eyes of the user.

6. The balance rehabilitation apparatus of claim 5, wherein when the display and optical frequency-flashed stimulation unit is in the frequency-flashed stimulation mode, the flashing image displayed by the display and optical frequency-flashed stimulation unit is flashed with a predetermined optical frequency, and the predetermined optical frequency is between 25 Hz and 50 Hz.

7. The balance rehabilitation apparatus of claim 5, wherein when the display and optical frequency-flashed stimulation unit is in the frequency-flashed stimulation mode, the flashing image displayed by the display and optical frequency-flashed stimulation unit stimulates eyes of the user with an optical frequency difference.

8. The balance rehabilitation apparatus of claim 5, wherein the display and optical frequency-flashed stimulation unit is switchable to a training mode, in the display mode, the virtual image and the display and optical frequency-flashed stimulation unit are moved in the same coordinate system, the virtual image is co-moved with the display and optical frequency-flashed stimulation unit and the user does not feel movement of the virtual image when the display and optical frequency-flashed stimulation unit is moved; in the training mode, the virtual image and the display and optical frequency-flashed stimulation unit are moved in different coordinate systems, and the virtual image is not moved and the user feels the virtual image moving in an opposite direction relative to the display and optical frequency-flashed stimulation unit when the display and optical frequency-flashed stimulation unit is moved.

9. The balance rehabilitation apparatus of claim 1, further including a training unit, wherein the training unit includes a balance board configured for the user to stand thereon, a control box electrically connected with the balance board and a monitor electrically connected with the control box, the balance board outputs a sense signal corresponding to a posture of the user, and the control box receives the sense signal and controls the monitor to display an image corresponding to the sense signal.

10. The balance rehabilitation apparatus of claim 1, further including a training unit, wherein the training unit includes a plurality of stepping boards spacedly disposed on a ground and configured for the user to step thereon, a control box electrically connected with the plurality of stepping boards and a monitor electrically connected with the control box, each of the plurality of stepping boards outputs a stepping signal corresponding to respective stepped states, and the control box receives said stepping signals and controls the monitor to display an image corresponding to the said stepping signals.

11. The balance rehabilitation apparatus of claim 1, wherein the training unit further includes a plurality of indicator lights respectively disposed on the plurality of stepping boards.

12. The balance rehabilitation apparatus of claim 1, further including a training unit, wherein the training unit includes a base, a vibration module disposed on the base, a vibration board disposed on the vibration module and a control box electrically connected with the vibration module, the vibration board is drivable to vibrate by the vibration module and configured for the user to stand thereon, and a vibration frequency of the vibration board is between 25 Hz and 250 Hz.

13. The balance rehabilitation apparatus of claim 1, further including a training unit, wherein the training unit includes at least one vibration belt generating vibrations and configured to be secured to the user, and a vibration frequency of the vibration belt is between 25 Hz and 250 Hz.

14. The balance rehabilitation apparatus of claim 1, further including a training unit, wherein the training unit includes a foot stimulation pad configured for the user to step thereon, and the foot stimulation pad includes a base portion and a plurality of foot stimulation portions protruding upward from the base portion.

15. The balance rehabilitation apparatus of claim 1, further including a protection unit, wherein the protection unit includes a safety strap configured for the user to wear and a fixed strap connected with the safety strap and configured to be connected with a fixed object above the user.
